(19) United States
(12) Patent Application Publication
(10) Pub. No.: US 2005/0033169 A1
(43) Pub. Date: Feb. 10, 2005

(54) Ultrasonic scanning method and an ultrasonic diagnostic apparatus

(76) Inventor: Kazuyoshi Hayasaka, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/891,676

(22) Filed: Jul. 15, 2004

(30) Foreign Application Priority Data
Jul. 22, 2003 (JP) 2003-277600

(51) Int. Cl. A61B 8/00

(52) U.S. Cl. 600/437

(57) ABSTRACT

An ultrasonic system and method for automatic control of the start of an interval scan without a burden on an operator and automatic control of interval period in an interval scan without performing a preliminary interval scan, an interval scan will be controlled using a sufficiently intense ultrasound to dissipate a contrast agent, by monitoring a dispersion or mean brightness value of pixels in an entire image or part thereof obtained by scanning for every short period of time with a weak ultrasound so as not to dissipate the contrast agent.

### BACKGROUND OF THE INVENTION

The present invention is related to an ultrasonic scanning method and an ultrasound diagnostic apparatus, more particularly to an ultrasonic scanning method and an ultrasound diagnostic apparatus, which allow automatic control of the start of an interval scan and automatic control of the interval period between interval scans without attempting a preliminary interval scan, with no additional burden on the operator.

The ultrasound diagnostic apparatus of the Prior Art requires an operator to set the interval period of time of the interval scan prior to injecting a contrast agent to the subject, then to instruct the start of an interval scan at an appropriate timing while observing the ultrasound image generated by a scan using a weak ultrasound so as not to dissipate the contrast agent, thereafter the apparatus performs an interval scan using a sufficiently intense ultrasound to dissipate the contrast agent at a predefined interval period of time.

It is possible, however, that the operator sets such a short interval that the contrast agent cannot be sufficiently recovered, or an excessively longer period of time than as required to sufficiently recover the contrast agent. There has therefore been proposed an automated setting of the appropriate interval period of time of the interval scans by transmitting several times a sufficiently intense ultrasound to dissipate the contrast agent while varying the interval between scans (e.g., see patent reference no. 1).

[Patent Reference 1] JP-A-2002-177269 ([0004], [0017])

In the Prior Art, there has been a problem that the operator is required to continue to carefully watch the ultrasound image so as to instruct the start of an interval scan at a timing presumed to be the optimal, resulting in an additional burden on the operator. Also, there has been a problem that a preliminary interval scan is required to be attempted in order to automate the interval period of time of an appropriate interval scan.

### SUMMARY OF THE INVENTION

The object of the present invention therefore is to provide an ultrasonic scanning method and an ultrasound diagnostic apparatus, which allow automatic control of the start of an interval scan, and automatic control of the interval period of time of an interval scan without need of a preliminary interval scan, without an additional burden on the operator.

In a first aspect, the present invention provides an ultrasonic scanning method characterized by controlling an interval scan using a sufficiently intense ultrasound to dissipate a contrast agent, based on a dispersion value or mean brightness value of pixels of an entire image or part thereof, obtained by scanning for every short period of time by use of a weak ultrasound so as not to dissipate the contrast agent.

The dispersion value or mean brightness value of pixels of an entire image or part thereof, obtained by scanning for every short period of time by use of a weak ultrasound so as not to dissipate the contrast agent, may increase along with the in-flow of the contrast agent into the imaging area, and may decrease along with the contrast agent flew out from the imaging area.

In the ultrasonic scanning method in accordance with the first aspect above, the interval scan using a sufficiently intense ultrasound to dissipate the contrast agent will be controlled by monitoring the dispersion value or mean brightness value of pixels of the entire image or part thereof, obtained by scanning for every short interval period of time using a weak ultrasound so as not to dissipate the contrast agent, according to the change of dispersion or brightness value. This allows automated control of the start of an interval scan or automated control of the interval period of time of an interval scan without need to attempt a preliminary interval scan.

In a second aspect, the present invention provides an ultrasonic scanning method according to the abovementioned arrangement, characterized by controlling the start of an interval scan using a sufficiently intense ultrasound to dissipate the contrast agent, based on the dispersion value or mean brightness value of pixels of the entire image or part thereof, obtained by scanning for every short period of time by use of the weak ultrasound so as not to dissipate the contrast agent.

The ultrasonic scanning method in accordance with the above described second aspect, the start of an interval scan using a sufficiently intense ultrasound to dissipate the contrast agent can be controlled by monitoring the dispersion value or mean brightness value of pixels of the entire image or part thereof, obtained by scanning for every short interval period of time using a weak ultrasound so as not to dissipate the contrast agent, for detecting the change in the dispersion or mean brightness value indicative of the in-flow of the contrast agent into the imaging area. This allows automatic control of the start of an interval scan without an additional burden on the operator.

In a third aspect, the present invention provides an ultrasonic scanning method according to the above arrangement, characterized by computing the dispersion value or mean brightness value of pixels of the entire image or part thereof, obtained by scanning for every short period of time by use of a weak ultrasound so as not to dissipate the contrast agent as a monitor value, for performing a first scan of the interval scan using a sufficiently intense ultrasound to dissipate the contrast agent when a monitor value exceeds a starting monitor value.

In the ultrasonic scanning method in accordance with the third aspect, the interval scan using a sufficiently intense ultrasound to dissipate the contrast agent can be started, by monitoring the dispersion or mean brightness value of pixels of the entire image or part thereof, obtained by scanning for every short interval period of time using a weak ultrasound so as not to dissipate the contrast agent, for detecting the increase of the dispersion or mean brightness value in excess of a start monitoring value indicative of the in-flow of the contrast agent into the imaging area. This allows automatic start of an interval scan without an additional burden on the operator.

In a fourth aspect, the present invention provides an ultrasonic scanning method according to the above arrangement, characterized by computing the starting monitor value by multiplying a coefficient value by the monitor value at the time of the instruction by an operator.

In the ultrasonic scanning method in accordance with the fourth aspect as described above, after the contrast agent is injected to the subject, upon an instruction of stand-by by an operator at an appropriate timing while watching the ultrasound image generated by the scan using a weak ultrasound so as not to dissipate the contrast agent to thereby perform an automatic configuration of the starting monitor value, an interval scan will be triggered when the dispersion value or mean brightness value exceeds the starting monitor value. As the change of monitor value at or around the timing of stand-by instruction is slower than the change of monitor value around the start timing of an interval scan, there will not be a problem if the instruction is given a little earlier or later. This allows an interval scan to start automatically without need to an additional burden on the operator.

In a fifth aspect, the present invention provides an ultrasonic scanning method according to the above arrangement, characterized by allowing the operator to set the starting monitor value.

In the ultrasonic scanning method in accordance with the fifth aspect as described above, after setting the starting monitor value and injecting the contrast agent to the subject by an operator, an interval scan will be started when the dispersion value or mean brightness value exceeds the starting monitor value. This dispenses with the need of giving an instruction to start by the operator at the starting timing of interval scan while closely watching the ultrasound image generated by the scan using a weak ultrasound so as not to dissipate the contrast agent, resulting in an automated start of an interval scan without an additional burden on the operator.

In a sixth aspect, the present invention provides an ultrasonic scanning method according to the above arrangement, characterized by controlling an interval between two interval scans using a sufficiently intense ultrasound to dissipate the contrast agent, based on the dispersion value or mean brightness value of pixels of the entire image or part thereof, obtained by scanning for every short period of time by use of a weak ultrasound so as not to dissipate the contrast agent.

In the ultrasonic scanning method in accordance with the sixth aspect above, the interval period of time of an interval scan using a sufficiently intense ultrasound to dissipate the contrast agent is controlled by monitoring the dispersion value or mean brightness value of pixels of the entire image or part thereof obtained by scanning for every short period of time by use of a weak ultrasound so as not to dissipate the contrast agent, and by detecting the velocity of in-flow of the contrast agent into the imaging area based on the dispersion or mean brightness value. This may eliminate the concern about the setting of such a short interval that the contrast agent cannot be sufficiently recovered or an excessively longer interval than as required to sufficiently recover the contrast agent. In addition, there is no need to attempt to perform a preliminary interval scan.

In a seventh aspect, the present invention provides an ultrasonic scanning method according to the above arrangement, characterized by computing the dispersion value or mean brightness value of pixels of the entire image or part thereof, obtained by scanning for every short period of time by use of the weak ultrasound so as not to dissipate the contrast agent as a monitor value, for performing a scan using a sufficiently intense ultrasound to dissipate the contrast agent when the monitor value having decreased at a previous scan using a sufficiently intense ultrasound to dissipate contrast agent exceeds a trigger monitor value.

In the ultrasonic scanning method in accordance with the seventh aspect as described above, a scan using a sufficiently intense ultrasound to dissipate the contrast agent is performed by monitoring the dispersion value or mean brightness value of pixels of an entire image or part thereof obtained by scanning for every short period of time by use of a weak ultrasound so as not to dissipate the contrast agent, and by detecting the recover of the contrast agent by virtue of the increase of the dispersion or mean brightness value having once decreased at the previous scan using a sufficiently intense ultrasound to dissipate the contrast agent. This allows automated and appropriate control of the interval period of an interval scan.

In an eighth aspect, the present invention provides an ultrasonic scanning method according to the above arrangement, characterized by allowing the operator to set the trigger monitor value.

In the ultrasonic scanning method in accordance with the eighth aspect, the interval period of time of an interval scan can be adjusted intentionally by an operator setting a triggering monitor value.

In a ninth aspect, the present invention provides an ultrasonic scanning method according to the above arrangement, characterized by performing a scan using a sufficiently intense ultrasound to dissipate the contrast agent when the monitor value having decreased at the previous scan using a sufficiently intense ultrasound to dissipate the contrast agent does not exceed the trigger monitor value after a predetermined waiting period has elapsed.

In the ultrasonic scanning method in accordance with the ninth aspect, the predetermined waiting period of time is the maximum value of the period of time of an interval scan. In other words, a scan using a sufficiently intense ultrasound to dissipate the contrast agent can be necessarily performed once for a predetermined waiting period.

In a tenth aspect, the present invention provides an ultrasound diagnostic apparatus, including an ultrasonic probe, ultrasonic scanning means for scanning inside a subject by means of the ultrasonic probe, ultrasonic imaging means for generating an ultrasonic image based on data obtained by the scan, monitor value obtaining means for computing the dispersion value or mean brightness value of an entire image or part thereof generated by scanning for every short period of time by use of a weak ultrasound so as not to dissipate the contrast agent, controlling means for controlling an interval scan using a sufficiently intense ultrasound to dissipate the contrast agent based on the computed monitor value, and ultrasonic image display means for displaying an ultrasonic image generated by the interval scan.

The ultrasound diagnostic apparatus in accordance with the tenth aspect as described above may allow the ultrasonic scanning method according to the first aspect above to be preferably effectuated.

In an eleventh aspect, the present invention provides an ultrasound diagnostic apparatus according to the above arrangement, in which the controlling means control the start of an interval scan using a sufficiently intense ultrasound to dissipate contrast agent, based on the monitor value.

The ultrasound diagnostic apparatus in accordance with the eleventh aspect may allow the ultrasonic scanning method according to the second aspect to be preferably effectuated.

In a twelfth aspect, the present invention provides an ultrasound diagnostic apparatus according to the above arrangement, in which the controlling means performs a first scan of the interval scan using a sufficiently intense ultrasound to dissipate the contrast agent when a monitor value exceeds a starting monitor value.

The ultrasound diagnostic apparatus in accordance with the twelfth aspect may allow the ultrasonic scanning method according to the third aspect to be preferably effectuated.

In a thirteenth aspect, the present invention provides an ultrasound diagnostic apparatus according to the above arrangement, further comprising starting monitor value computing means for computing the starting monitor value by multiplying a coefficient value by the monitor value at the time of an instruction by an operator.

The ultrasound diagnostic apparatus in accordance with the thirteenth aspect as described above may allow the ultrasonic scanning method according to the fourth aspect to be preferably effectuated.

In a fourteenth aspect, the present invention provides an ultrasound diagnostic apparatus according to the above arrangement, further comprising operating means for the operator to set the starting monitor value.

The ultrasound diagnostic apparatus in accordance with the fourteenth aspect may allow the ultrasonic scanning method according to the fifth aspect to be preferably effectuated.

In a fifteenth aspect, the present invention provides an ultrasound diagnostic apparatus according to the above arrangement, characterized by the controlling means controlling an interval period between two interval scans using a sufficiently intense ultrasound to dissipate the contrast agent, based on the monitor value.

The ultrasound diagnostic apparatus in accordance with the fifteenth aspect may allow the ultrasonic scanning method according to the sixth aspect to be preferably effectuated.

In a sixteenth aspect, the present invention provides an ultrasound diagnostic apparatus according to the above arrangement, characterized by the controlling means performing a scan using a sufficiently intense ultrasound to dissipate the contrast agent when the monitor value having decreased at a previous scan using a sufficiently intense ultrasound to dissipate the contrast agent exceeds a trigger monitor value.

The ultrasound diagnostic apparatus in accordance with the sixteenth aspect as described above may allow the ultrasonic scanning method according to the seventh aspect to be preferably effectuated.

In a seventeenth aspect, the present invention provides an ultrasound diagnostic apparatus according to the above arrangement, further comprising operating means for the operator to set the trigger monitor value.

The ultrasound diagnostic apparatus in accordance with the seventeenth aspect as described above may allow the ultrasonic scanning method according to the eighth aspect to be preferably effectuated.

In an eighteenth aspect, the present invention provides an ultrasound diagnostic apparatus according to the above arrangement, characterized by the controlling means performing a scan using a sufficiently intense ultrasound to dissipate the contrast agent when the monitor value having decreased at the previous scan using a sufficiently intense ultrasound to dissipate the contrast agent does not exceed the trigger monitor value after a predetermined waiting period has elapsed.

The ultrasound diagnostic apparatus in accordance with the eighteenth aspect may allow the ultrasonic scanning method according to the ninth aspect to be preferably effectuated.

In accordance with the ultrasonic scanning method and ultrasound diagnostic apparatus of the present invention, the start timing and interval of the interval scan may be automatically controlled corresponding to the situation of imaging.

Namely, the ultrasonic scanning method and ultrasound diagnostic apparatus in accordance with the present invention may allow automatic control of the start of an interval scan without an additional burden on the operator or automatic control of the interval period of time of an interval scan without attempt to perform a preliminary interval.

Further objects and advantages of the present invention will be apparent from the following description of the preferred embodiments of the invention as illustrated in the accompanying drawings.

### BRIEF DESCRIPTION OF THE DRAWINGS

FIG. 1 shows a schematic block diagram of an ultrasound diagnostic apparatus in accordance with the first preferred embodiment of the present invention;

FIG. 2 shows a schematic diagram illustrating the computing area of monitor value;

FIG. 3 shows a flowchart illustrating the protocol of an interval scan processing in the ultrasound diagnostic apparatus in accordance with the first preferred embodiment of the present invention;

FIG. 4 shows a schematic diagram illustrating the start timing and interval of interval scan in a situation in which the contrast agent rapidly flows in to the imaging area;

FIG. 5 shows a schematic diagram illustrating the interval period of interval scan in a situation in which the contrast agent hardly flows in to the imaging area.

### DETAILED DESCRIPTION OF THE INVENTION

In the following, preferred embodiments in accordance with the present invention will be described with reference to the accompanying drawings.

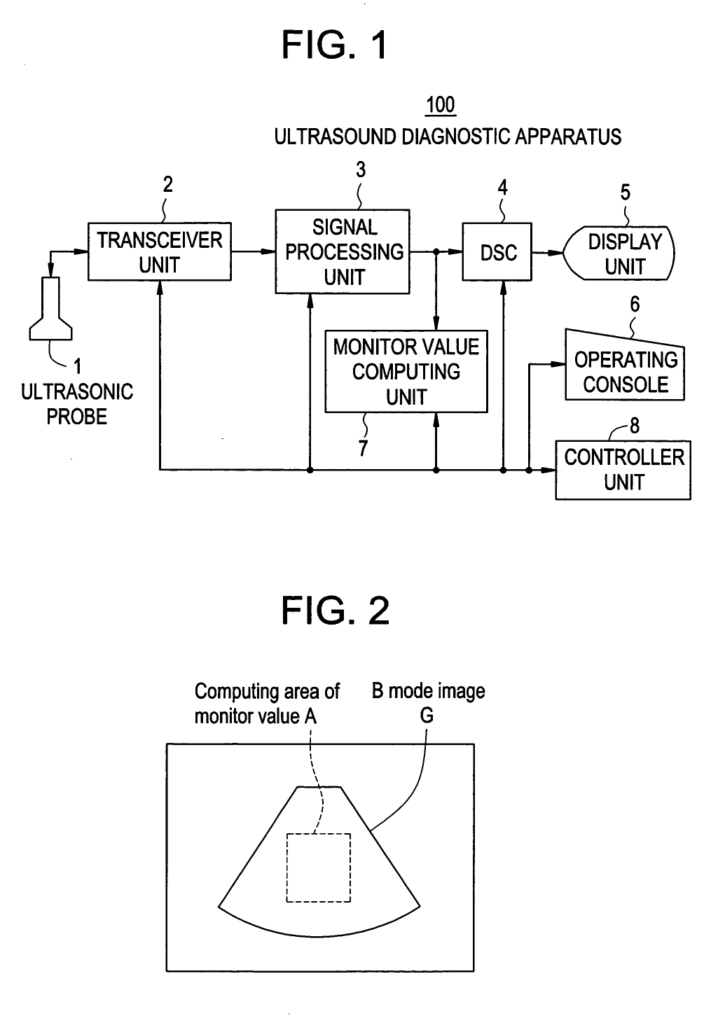

FIG. 1 shows a schematic block diagram of an ultrasound diagnostic apparatus 100 in accordance with the first preferred embodiment of the present invention.

This ultrasound diagnostic apparatus 100 includes an ultrasonic probe 1, a transceiver unit 2 for driving the ultrasonic probe 1 to scan a subject with the ultrasound to output received signals, a signal processing unit 3 for generating an ultrasonic image data based on the received signals, a DSC (Digital Scan Converter) 4 for generating display image data based on the ultrasonic image data, a display unit 5 for displaying an image based on the display image data, an operating console 6 for an operator to input an instruction, a monitor value computing unit 7 for computing a dispersion value or mean brightness value as a monitor value based on the ultrasonic image data, and a controller unit 8 for controlling the operation of the entire apparatus.

As shown in FIG. 2, an operator can set a monitor value computing area A on a B mode image G prior to an interval scan processing. The entire B mode image G can be set as the monitor value computing area A.

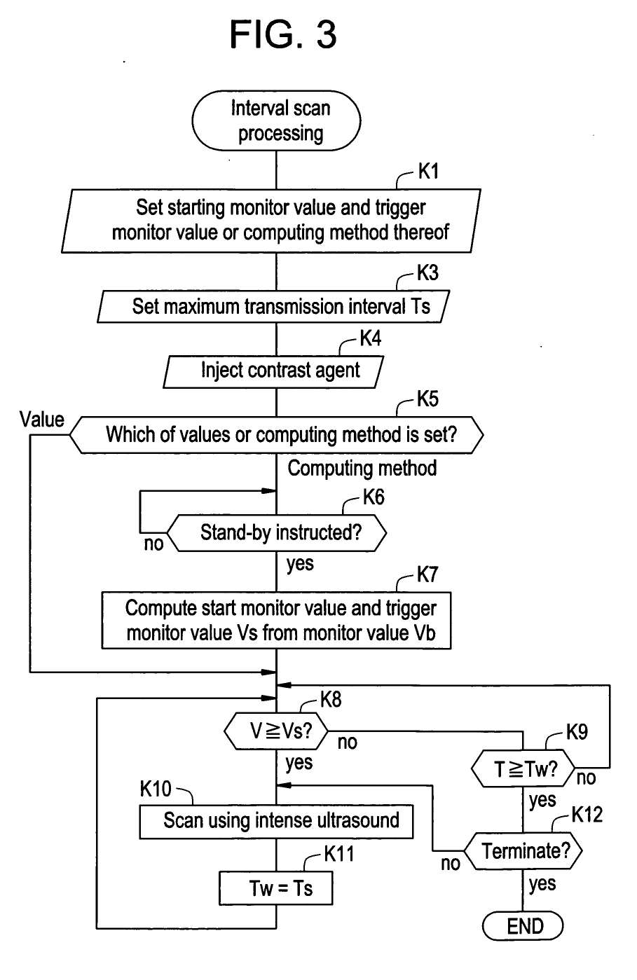

FIG. 3 shows a flowchart illustrating the protocol of an interval scan processing in the ultrasound diagnostic apparatus 100.

In parallel to the interval scan, it is assumed that the ultrasound diagnostic apparatus 100 generates sequentially B mode images (for example, a frame rate between 50 and 10) by scanning for every short period of time (for example, 20 ms to 100 ms) using a weak ultrasound so as not to dissipate the contrast agent, to compute a dispersion or mean brightness value of the monitor value computing area A in the latest B mode image as the monitor value.

In step K1, the operator can set either a starting monitor value and trigger monitor value, or set the computing method of the starting monitor value and trigger monitor value. The value or computing method of those starting monitor value and trigger monitor value may be identical or different. In this example a computing method is set such that “start monitor value=trigger monitor value is substituted with a value Vs=V×□, or the value that is the monitor value V multiplied by □ when the stand-by instruction is given by the operator”.

In step K3, the operator will set the maximum transmission interval Ts.

In step K4, the operator will inject the contrast agent to the subject.

In step K5, the controller unit 8 proceeds the process to step K8 if the start monitor value and trigger monitor value is set, or proceeds the process to step K6 if the computing method of start monitor value and trigger monitor value is set.

In step K6, the controller unit 8 will wait until a stand-by instruction is given by the operator, and will proceed to step K6 when the stand-by instruction is given.

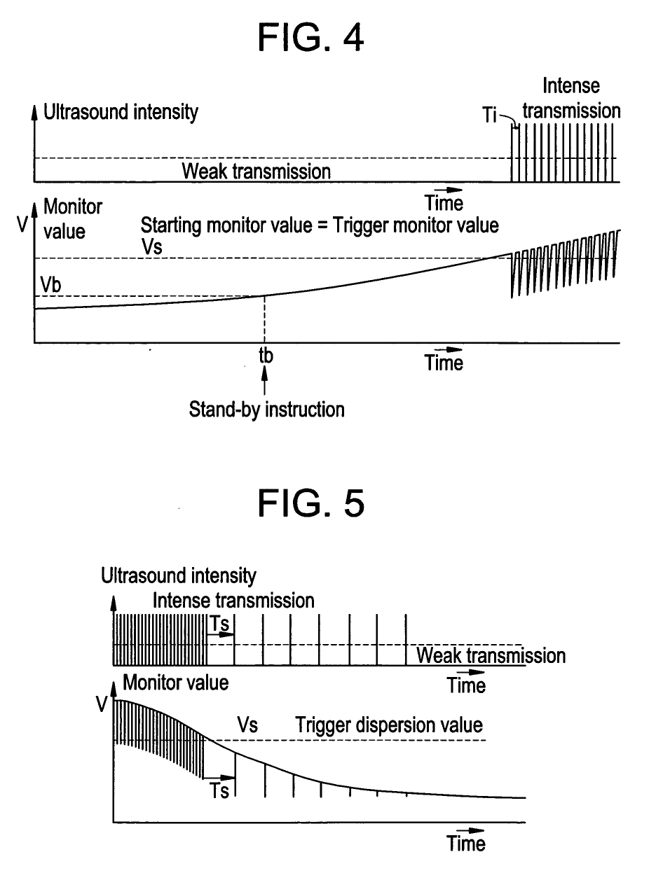

The operator will direct a stand-by instruction at an appropriate timing while watching the B mode image G by the weak ultrasound. In this example, the stand-by instruction assumes to be given at the time tb, as shown in FIG. 4.

In step K7, the controller unit 8 will compute the starting monitor value and triggering monitor value Vs from the monitor value Vb at the time tb (or immediately thereafter), as shown in FIG. 4.

In step K8, the controller unit 8 will proceed to step K9 if the most current monitor value V is not equal to or more than the start monitor value Vs, otherwise proceed to step K10 if equal to or more than the start monitor value Vs.

In step K9, the process will go back to step K8 if the elapsed time T is not equal to or more than a waiting time Tw, otherwise proceed to step K12 if equal to or more than the waiting time Tw.

At this point the elapsed time T is indicative of the elapsed time from the beginning of the interval scan process prior to the first scan of the interval scan using a sufficiently intense ultrasound to dissipate the contrast agent, and is indicative of the elapsed time since the immediately preceding scan using a sufficiently intense ultrasound to dissipate the contrast agent when the first scan of the interval scan using a sufficiently intense ultrasound to dissipate the contrast agent has been performed. The waiting time Tw is initially set to a sufficiently long time (for example, 60 seconds) prior to the first scan of the interval scan using a sufficiently intense ultrasound to dissipate the contrast agent, and will be set to the maximum transmission interval Ts after the first scan of the interval scan using a sufficiently intense ultrasound to dissipate the contrast agent will have been performed.

In step K10, the ultrasound diagnostic apparatus 100 will perform a scan using a sufficiently intense ultrasound to dissipate the contrast agent.

In step K11, the waiting time Tw will be set to the maximum transmission interval Ts. Then the process will return to step K8.

In step K12, the controller unit 8 will terminate the process if the operator indicates “terminate”, or will proceed to step K10 if the operator does not indicate “terminate”.

FIG. 4 shows a situation in which the contrast agent flows rapidly in to the imaging area.

After instructing a stand-by, if the monitor value V exceeds the start monitor value Vs, first scan of the interval scan using a sufficiently intense ultrasound to dissipate the contrast agent will be performed. Then each time the monitor value, which has been decreased along with the dissipation of the contrast agent due to the intense ultrasound, is rapidly recovered beyond the trigger monitor value Vs, another scan using a sufficiently intense ultrasound to dissipate the contrast agent will be performed. Thus the interval time of the interval scan Ti will become shorter (for example, Ti=100 ms).

FIG. 5 shows a situation in which the contrast agent does not almost flow in to the imaging area.

Since the monitor value, which has been decreased by the dissipation of the contrast agent due to the intense ultrasound is not recovered beyond the triggering monitor value Vs after having elapsed the maximum transmission interval Ts, a scan using a sufficiently intense ultrasound to dissipate the contrast agent will be performed at almost every maximum transmission interval Ts (for example, Ts=2s).

Many widely different embodiments of the invention may be constructed without departing from the spirit and the scope of the present invention. It should be understood that the present invention is not limited to the specific embodiments described in the specification, except as defined in the appended claims.

1. An ultrasonic scanning method, comprising the step of controlling an interval scan using a sufficiently intense ultrasound to dissipate a contrast agent, based on a dispersion value or mean brightness value of pixels of an entire image or part thereof, obtained by scanning for every short period of time by use of a weak ultrasound so as not to dissipate the contrast agent.

2. The ultrasonic scanning method according to claim 1, comprising the step of controlling the start of an interval scan using a sufficiently intense ultrasound to dissipate the contrast agent, based on the dispersion value or mean brightness value of pixels of the entire image or part thereof, obtained by scanning for every short period of time by use of the weak ultrasound so as not to dissipate the contrast agent.

3. The ultrasonic scanning method according to claim 2, comprising the steps of: computing the dispersion value or mean brightness value of pixels of the entire image or part thereof, obtained by scanning for every short period of time by use of the weak ultrasound so as not to dissipate the contrast agent as a monitor value; and performing a first scan of the interval scan using a sufficiently intense ultrasound to dissipate the contrast agent when a monitor value exceeds a starting monitor value.

4. The ultrasonic scanning method according to claim 3, comprising the step of computing the starting monitor value by multiplying a coefficient value by the monitor value at the time of an instruction by an operator.

5. The ultrasonic scanning method according to claim 3, comprising the step of allowing the operator to set the starting monitor value.

6. The ultrasonic scanning method according to claim 1, comprising the step of controlling an interval between two interval scans using a sufficiently intense ultrasound to dissipate the contrast agent, based on the dispersion value or mean brightness value of pixels of the entire image or part thereof, obtained by scanning for every short period of time by use of a weak ultrasound so as not to dissipate the contrast agent.

7. The ultrasonic scanning method according to claim 6, comprising the steps of: computing the dispersion value or mean brightness value of pixels of the entire image or part thereof, obtained by scanning for every short period of time by use of the weak ultrasound so as not to dissipate the contrast agent as a monitor value; and performing a scan using a sufficiently intense ultrasound to dissipate the contrast agent when the monitor value having decreased at a previous scan using a sufficiently intense ultrasound to dissipate contrast agent exceeds a trigger monitor value.

8. The ultrasonic scanning method according to claim 7, comprising the step of allowing the operator to set the trigger monitor value.

9. The ultrasonic scanning method according to claim 7, comprising the step of performing a scan using a sufficiently intense ultrasound to dissipate the contrast agent when the monitor value having decreased at the previous scan using a sufficiently intense ultrasound to dissipate the contrast agent does not exceed the trigger monitor value after a predetermined waiting period has elapsed.

10. An ultrasound diagnostic apparatus, comprising:
an ultrasonic probe;
an ultrasonic scanning device for scanning inside a subject by means of the ultrasonic probe;
an ultrasonic imaging device for generating an ultrasonic image based on data obtained by the scan;
a monitor value obtaining device for computing the dispersion value or mean brightness value of an entire image or part thereof generated by scanning for every short period of time by use of a weak ultrasound so as not to dissipate the contrast agent as a monitor value;
a controlling device for controlling an interval scan using a sufficiently intense ultrasound to dissipate the contrast agent based on the computed monitor value; and
an ultrasonic image display device for displaying an ultrasonic image generated by the interval scan.

11. The ultrasound diagnostic apparatus according to claim 10, wherein said controlling device control the start of an interval scan using a sufficiently intense ultrasound to dissipate the contrast agent, based on the monitor value.

12. The ultrasound diagnostic apparatus according to claim 11, wherein said controlling device performs a first scan of the interval scan using a sufficiently intense ultrasound to dissipate the contrast agent when a monitor value exceeds a starting monitor value.

13. The ultrasound diagnostic apparatus according to claim 12, further comprising a starting monitor value computing device for computing the starting monitor value by multiplying a coefficient value by the monitor value at the time of an instruction by an operator.

14. The ultrasound diagnostic apparatus according to claim 12, further comprising an operating device for the operator to set the starting monitor value.

15. The ultrasound diagnostic apparatus according to claim 10, wherein said controlling device controls an interval period between two interval scans using a sufficiently intense ultrasound to dissipate the contrast agent, based on the monitor value.

16. The ultrasound diagnostic apparatus according to claim 15, wherein said controlling device performs a scan using a sufficiently intense ultrasound to dissipate the contrast agent when the monitor value having decreased at a previous scan using a sufficiently intense ultrasound to dissipate the contrast agent exceeds a trigger monitor value.

17. An ultrasound diagnostic apparatus according to claim 16, further comprising an operating device for the operator to set the trigger monitor value.

18. The ultrasound diagnostic apparatus according to claim 16, wherein said controlling device performs a scan using a sufficiently intense ultrasound to dissipate the contrast agent when the monitor value having decreased at the previous scan using a sufficiently intense ultrasound to dissipate the contrast agent does not exceed the trigger monitor value after a predetermined waiting period has elapsed.
